(19) United States
(12) Patent Application Publication
(10) Pub. No.: US 2012/0064020 A1
(43) Pub. Date: Mar. 15, 2012

(54) NOVEL COMPOSITION

(76) Inventors: Mathias Gempler, Pfeffingen (CH); Remo Graeub, Bern (CH); Leo Hochegger, Ormalingen (CH); Dominik Imfeld, Munchenstein (CH); Heidi Moser, Bubendorf (CH)

(21) Appl. No.: 13/201,996

(22) PCT Filed: Feb. 16, 2010
(86) PCT No.: PCT/EP2010/000943

§ 371 Date: Nov. 30, 2011

Related U.S. Application Data
(60) Provisional application No. 61/202,341, filed on Feb. 20, 2009.
(60) Provisional application No. 61/202,342, filed on Feb. 20, 2009.

(30) Foreign Application Priority Data
Feb. 20, 2009 (EP) 09153270.5
Feb. 20, 2009 (EP) 09153271.3

(51) Int. Cl. A61K 36/8998; A61P 17/00; A61Q 1/02; A61K 8/97

(52) U.S. Cl. 424/63; 424/750

(57) ABSTRACT

The present invention relates to a composition comprising N2-(1-oxohexadecyl)-lysyl-valyl-lysine or a salt thereof, spent grain wax and/or conjugated linoleic acid. The compositions are particularly useful for the treatment or co-treatment of rosacea and its symptoms. Furthermore the invention relates to a stable W/O emulsion pre-mix comprising the composition according to the invention.

The present invention relates to a composition comprising N2-(1-oxohexadecyl)-lysyl-valyl-lysine or a salt thereof, spent grain wax and/or conjugated linoleic acid. The compositions are particularly useful for the treatment or co-treatment of rosacea and its symptoms. Furthermore the invention relates to a stable W/O emulsion pre-mix comprising a composition according to the invention.

Rosacea develops gradually starting as frequent blushing and frequent irritation of the facial skin. More advanced rosacea is characterized by a vascular stage where patients display increasingly severe erythema (abnormal redness of the skin) and telangiectasia (visible red lines due to abnormal dilatation of capillary vessels and arterioles). Pimple-like eruptions, which may be solid (called papules or nodules) or puss filled (known as pustules) may develop. Such eruptions often look like acne, but whiteheads or blackheads (common symptoms of acne) are not normally present. Later-stage rosacea is characterized by rhinophyma (enlargement of the nose). If left untreated, rosacea can progress to irreversible disfigurement. Rosacea symptoms are often aggravated by sun exposure, changes or extremes in temperature, wind, and consumption of certain foods, such as spicy foods, caffeine, and alcohol.

There is no known cure for rosacea. Current treatments, which are directed to control of redness, inflammation, and skin eruptions, are of limited effectiveness in many patients and, generally, can be used only for a limited duration.

Antibiotics are the traditional first line of therapy. Long-term treatment (5 to 8 weeks or more) with oral antibiotics such as tetracycline, minocycline, doxycycline or clarithromycin may control skin eruptions. Alternative oral treatments include vitamin A medications, such as isoretinoin and antifungal medications. Unfortunately, such oral medications often cause side effects and many people have limited tolerance. Topical treatments, such at topically applied antibiotics and antifungals (such as metronidazole) or steroids, are available but also have limited effectiveness, severe side effects and cannot treat all symptoms.

Thus, there remains a need for topical compositions for the treatment of rosacea and its symptoms having little to no side effects, in particular for topical cosmetic treatments which can be used on a daily basis.

Surprisingly, it has been found that a composition comprising N2-(1-oxohexadecyl)-lysyl-valyl-lysine, further comprising spent grain wax and/or conjugated linoleic acid is able to significantly ameliorate the symptoms caused by rosacea such as in particular subtype I rosacea (erythematotelangiectatic rosacea), most in particular skin redness, blushing and telangiectasia.

Furthermore, it has been found that the combination of spent grain wax and CLA acts synergistically on the expression of VEGF which translates into a significant reduction of reddening of the face and telangiectasia.

Thus, in a first embodiment, the invention relates to a composition comprising N2-(1-oxohexadecyl)-lysyl-valyl-lysine, spent grain wax and/or conjugated linoleic acid i.e. a composition comprising at least two compounds selected from N2-(1-oxohexadecyl)-lysyl-valyl-lysine, spent grain wax and conjugated linoleic acid. Particularly preferred are compositions comprising N2-(1-oxohexadecyl)-lysyl-valyl-lysine, spent grain wax and conjugated linoleic acid.

In particular embodiment the composition is a pre-mix comprising 0.0001-1 wt.-%, preferably 0.01 to 0.05 wt.-% of N2-(1-oxohexadecyl)-lysyl-valyl-lysine, 20-60 wt.-%, preferably 30-50 wt.-% of spent grain wax and/or from 1-30 wt.-%, preferably 15-25 wt.-% of conjugated linoleic acid and optionally 1-10 wt.-%, preferably 4-7 wt.-% water and/or 5-35 wt.-%, preferably 10-20wt.-% of glycerin.

In a particular preferred embodiment the composition is a pre-mix comprising

i) 0.0001-1 wt.-%, preferably 0.01 to 0.05 wt.-% of N2-(1-oxohexadecyl)-lysyl-valyl-lysine

ii) 20-60 wt.-%, preferably 30-50 wt.-% of spent grain wax and/or 1-30 wt.-%, preferably 15-25 wt.-% of conjugated linoleic acid; optionally further comprising

iii) 1-10 wt.-%, preferably 4-7 wt.-% water and/or 5-35 wt.-%, preferably 10-20wt.-% of glycerin.

Further cosmetically acceptable ingredients such as e.g. antioxidants, preservatives, stabilisators may also be present in the pre-mix in amounts of a total of up to 20 wt.-%, wherein the total amount of the ingredients sums up to 100 wt.-%. Preferably, water and glycerin are present in the pre-mix.

Thus, in a further particular embodiment the invention relates to a pre-mix comprising from 0.01 to 0.05 wt.-% of N2-(1-oxohexadecyl)-lysyl-valyl-lysine, from 30-50 wt.-% of spent grain wax and from 15-25 wt.-% of conjugated linoleic acid, 1-10 wt.-% water and 5-35 wt.-% of glycerin.

Surprisingly, it has been found that behenic acid is able to stabilize the above mentioned pre-mix in form of a W/O emulsion. Thus, in a specific embodiment, the pre-mix comprises next to water and glycerin an effective amount of behenic acid in order to obtain a stable product form suitable for commercial purposes. The behenic acid is preferably present in an amount of 3-10 wt.-% such as 3-7 wt.-%, preferably 5 to 6 wt.-%, based on the total weight of the pre-mix.

Thus in a further particular embodiment, the pre-mix comprises about 0.01-1 wt.-% of N2-(1-oxohexadecyl)-lysyl-valyl-lysine, 25-50 wt.-% of spent grain wax, 10-25 wt.-%, of conjugated linoleic acid, 1-10 wt.-% of water, 5-35 wt.-% of glycerin and 3-10 wt.-% of behenic acid such as in particular about 0.01 to 0.05 wt.-% of N2-(1-oxohexadecyl)-lysyl-valyl-lysine, 30-50 wt.-% of spent grain wax and 15-25 wt.-% of conjugated linoleic acid, 1-10 wt.-% of water, 5-35 wt.-% of glycerin and from 3-7 wt.-% of behenic acid.

In a particular preferred embodiment, the pre-mix comprises about 0.01-0.03 wt.-% of N2-(1-oxohexadecyl)-lysyl-valyl-lysine, 38-42 wt.-% of spent grain wax, 18-22 wt.-% of conjugated linoleic acid, 5.5-6.5 wt.-% of water, 13-15 wt.-% of glycerin and 5-6 wt.-% of behenic acid.

In all embodiments, the N2-(1-oxohexadecyl)-lysyl-valyl-lysine or a salt thereof is preferably the bistrifluoroacetate salt of N2-(1-oxohexadecyl)-L-lysyl-L-valyl-L-lysine (listed in the CTFA Dictionary as Palmitoyl Tripeptide-5, CAS-No 623172-56-5) which is commercially available at DSM Nutritional Products Ltd. under the trade name SYN®-COLL (aqueous, unpreserved, glycerin based solution of 900-1100 ppm Palmitoyl Tripeptide-5).

Conjugated linoleic acid (hereinafter referred to also as CLA) comprises a group of positional and geometric isomers of linoleic acid in which various configurations of cis and trans double bonds at positions (6,8), (7,9), (8,10), (9,11), (10,12) or (11,13) are possible. Thus, twenty-four different isomers of CLA exist.

The invention also includes derivatives of the free acid which thus comprise conjugated linoleic acid moieties. Preferable derivatives include those derived from substitution of the carboxyl group of the acid, such as esters (e.g. retinyl esters, triglyceride esters, monoglyceride esters, diglyceride esters, phosphoesters), amides (e.g. ceramide derivatives), salts (e.g. alkali metal and alkali earth metal salts, ammonium salts); and/or those derived from substitution of the C18 carbon chain, such as alpha hydroxy and/or beta hydroxy derivatives.

In the case of triglyceride ester derivatives, all positional isomers of CLA substituents on the glycerol backbone are included. The triglycerides must contain at least one CLA moiety. For example, of the three esterifiable positions on the glycerol backbone, the 1 and 2 positions may be esterified with CLA and by another lipid at position 3 or, as an alternative, the glycerol backbone could be esterified by CLA at the 1 and 3 positions with another lipid at position 2.

Wherever the term “conjugated linoleic acid” or “CLA” is used in this specification it is to be understood that the derivatives thereof comprising CLA moieties are also included.

It is also possible to use a combination of two or more isomers of CLA and such combinations are within the scope of the present invention. Among all positional and geometrical isomers of CLA, the ones, which are particularly preferred, are cis-9-trans-11 and trans-10-cis-12 isomers. Most preferred are the cis-9-trans-11 and trans-10-cis-12 isomers in their free acid form.

Commercially CLA is e.g. available as Conjugated Linoleic Acid from Bioriginal, Netherlands.

Spent grain wax [CAS No. 97660-18-9] is derived from spent barley grains produced in the brewing process during beer wort production. In wort production for brewing beer, barley is cleaned and watered, and germinate in about a week. The germination process is halted by heating the barley in a malt kiln. The malt is then crushed and fresh brewing water is added and warmed. The mixture degrades through an enzyme reaction into beer wort. At a predetermined point of degradation, the process is stopped, and the barley grains are removed and dried for extraction of lipophilic constituents. Spent grain wax is extracted through a supercritical carbon dioxide extraction process at sixty degrees centigrade in an oxygen free environment. Spent grain wax contains naturally occurring fatty acids, vitamins and phytosterols. Further information on spent grain wax can also be found in Cosmetics & Toiletries (1990), 105(11), 59-62.

Commercially spent grain wax is e.g. available as Treberextrakt from Aromtech.

In another embodiment the composition according to the invention is a topical preparation further comprising cosmetically acceptable carrier. Such topical preparations are in particular suitable for the treatment or co-treatment of rosacea and its symptoms, such as in particular subtype I rosacea (erythematotelangiectatic rosacea), particularly skin redness, blushing and telangiectasia as well as for treatment or co-treatment of blotchy skin, sensitive skin, dry skin, irritated skin, inflamed skin, atopic skin.

The topical preparations are preferably prepared by incorporating an ‘effective amount’ of the active ingredients (as such) or of a pre-mix as outlined above into a cosmetically acceptable carrier.

The term ‘effective amount’ refers to an amount necessary to obtain a physiological effect and can be easily assessed by a person skilled in the art.

Preferably, the topical preparations according to the invention comprise about 0.00002 to 0.002 wt.-% of palmitoyl tripeptide-5, 0.04 to 4 wt.-% of conjugated linoleic acid and 0.02 to 2 wt.-% of spent grain wax based on the total weight of the topical preparation.

If a pre-mix is used, preferably an amount of at least 0.01% based on the total weight of the topical preparation is incorporated. More preferably, the topical preparations contain the pre-mix according to the invention in an amount of 0.1 to 10 wt-%, more preferably in amount from 0.5 to 5 wt.-% based on the total weight of the topical preparation.

The term “topical preparation” as used herein refers in particular to cosmetic compositions that can be topically applied to mammalian keratinous tissue such as e.g. human skin or hair (including eyelashes, the eyebrows) or the nails, particularly human skin.

The term “cosmetic composition” as used in the present application refers to cosmetic compositions as defined under the heading “Kosmetika” in Römpp Lexikon Chemie, 10th edition 1997, Georg Thieme Verlag Stuttgart, New York as well as to cosmetic compositions as disclosed in A. Domsch, “Cosmetic Compositions”, Verlag fur chemische Industrie (ed. H. Ziolkowsky), 4^{th} edition, 1992.

The term cosmetically acceptable carrier refers to all carriers and/or excipients and/or diluents conventionally used in topical compositions or compositions.

Preferably, the topical preparations are in the form of a suspension or dispersion in solvents or fatty substances, or alternatively in the form of an emulsion or micro emulsion (in particular of O/W- or W/O-type), PIT-emulsion, multiple emulsion (e. g. O/W/O- or W/O/W-type), pickering emulsion, hydrogel, alcoholic gel, lipogel, one- or multiphase solution or vesicular dispersion or other usual forms, which can also be applied by pens, as masks or as sprays. If the topical composition is or comprises an emulsion it can also contain one or more anionic, nonionic, cationic or amphoteric surfactant(s).

Preferred topical preparations are skin care compositions, and functional compositions.

Examples of skin care compositions are, in particular, body oils, body lotions, body gels, treatment creams, skin protection ointments, shaving compositions, such as shaving foams or gels, skin powders such as baby powder, moisturizing gels, moisturizing sprays, revitalizing body sprays, cellulite gels, face and/or body moisturizers, facial and/or body cleansers, face masks, anti acne compositions and/or peeling compositions.

Examples of functional compositions are cosmetic or pharmaceutical compositions containing active ingredients such as hormone compositions, vitamin compositions, vegetable extract compositions, anti-ageing compositions, and/or antimicrobial (antibacterial or antifungal) compositions without being limited thereto.

Topical preparations in accordance with the invention can be in the form of a liquid, lotion, a thickened lotion, a gel, a cream, a milk, an ointment, a paste, a powder, a make-up, or a solid tube stick and can be optionally be packaged as an aerosol and can be provided in the form of a mousse such as a aerosol mousse, a foam or a spray foam, a spray, a stick, a plaster, a cleanser, a soap, a wipe or a lyophilizate (such as the Pentapharm Dual Vial system).

The topical preparations according to the invention are preferably formulated as an oil-in-water or water-in-oil emulsion, water-in-silicone or silicone-in-water emulsion or as an aqueous serum or aqueous gel in particular in as an oil-in water emulsion (O/W emulsion).

The cosmetic preparations according to the invention have a pH in the range of 3-10, preferably in the range of pH of 4-8, most preferred in the range of pH 4-6.

In accordance with the present invention, the topical preparation may optionally comprise further ingredients such as ingredients for skin lightening; tanning prevention; treatment of hyperpigmentation; preventing or reducing acne, wrinkles, lines, atrophy and/or inflammation; as well as topical anesthetics; antimicrobial and/or antifungal agents; chelators and/or sequestrants; anti-cellulites and slimming (e.g. phytanic acid), firming, moisturizing and energizing, self tanning, soothing, as well as agents to improve elasticity and skin barrier and/or UV-filter substances. The topical cosmetic preparations can also contain usual cosmetic adjuvants and additives, such as preservatives/antioxidants, fatty substances/oils, water, organic solvents, silicones, thickeners, softeners, emulsifiers, antifoaming agents, moisturizers, aesthetic components such as fragrances, surfactants, fillers, sequestering agents, anionic, cationic, nonionic or amphoteric polymers or mixtures thereof, propellants, acidifying or basifying agents, dyes, colorings/colorants, abrasives, absorbents, essential oils, skin sensates, astringents, antifoaming agents, pigments or nanopigments, e.g. those suited for providing a photoprotective effect by physically blocking out ultraviolet radiation, or any other ingredients usually formulated into cosmetic compositions. Such cosmetic ingredients commonly used in the skin care industry, which are suitable for use in the topical preparations of the present invention are e.g. described in the CTFA Cosmetic Ingredient Handbook, Second Edition (1992) without being limited thereto.

The usual cosmetic adjuvants and additives such as e.g. emulsifiers, thickeners, surface active ingredients and film formers can show synergistic effects which can be determined by the expert in the field with normal trials, or with the usual considerations regarding the formulation of cosmetic composition.

The necessary amounts can, based on the desired product, easily be determined by the skilled person. The cosmetically active ingredients useful herein can in some instances provide more than one benefit or operate via more than one mode of action.

If nothing else is stated, the carrier, excipients, additives, diluents, adjuvant and additives etc. mentioned in the following are in particular suitable for topical preparations according to the present invention.

The topical preparations according to the present invention may contain further cosmetically active ingredients. Examples of cosmetically active ingredients comprise peptides (e.g., Matrixyl™ [pentapeptide derivative], one or both of the peptides contained in SYN®-TACKS from DSM Nutritional Products Ltd., Branch Pentapharm), oligopeptides, wax-based synthetic peptides and palmitoyl-oligopeptide), iodopropyl butylcarbamate, glycerol, urea, guanidine (e.g. amino guanidine); vitamins and derivatives thereof such as vitamin C (ascorbic acid), vitamin A (e.g., retinoid derivatives such as retinyl palmitate or retinyl propionate), vitamin E (e.g., tocopherol acetate), vitamin B₃ (e.g. niacinamide) and vitamin B₅ (e.g. panthenol), vitamin B₆ and vitamin B₁₂, biotin, folic acid; anti-acne actives or medicaments (e.g. resorcinol, salicylic acid, and the like); antioxidants (e.g. phytosterols, lipoic acid); flavonoids (e.g. isoflavones, phytoestrogens); skin soothing and healing agents such as aloe vera extract, allantoin and the like; agents suitable for aesthetic purposes such as essential oils, fragrances, skin sensates, opacifiers, aromatic compounds (e.g., clove oil, menthol, camphor, eucalyptus oil, and eugenol and their derivatives), desquamatory actives, hydroxy acids such as AHA acids, BHA acids, poly unsaturated fatty acids, radical scavengers, farnesol, antifungal actives in particular bisabolol, alkyldiols such as 1,2-pentanediol, hexanediol or 1,2-octanediol, phytol, polyols such as phytanetriol, ceramides and pseudoceramides, amino acids, protein hydrolysates, polyunsaturated fatty acids, plant extracts like kinetin, DNA or RNA and their fragmentation products, carbohydrates, conjugated fatty acids, carnitin, carnosine, biochinonen, phytofluen, phytoen, and their corresponding derivatives, co-enzyme Q10/ubiquinone), anti-oxidants [preferably (−)-epigallocatechin gallate (EGCG), hydroxytyrosol, and/or olive extract, shea butter, algae extract, cocoa butter, aloe extract and elastin without being limited thereto.

Preferred examples of cosmetically active ingredients are vitamin C (ascorbic acid) and/or its derivatives (e.g. ascorbyl phosphate such as Stay C (sodium ascorbyl monophosphate) from DSM Nutritional Products Ltd.), vitamin A and/or its derivatives (e.g., retinoid derivatives such as retinyl palmitate or retinyl propionate), vitamin E and/or its derivatives (e.g., tocopherol acetate), vitamin B₆, vitamin B₁₂, biotin, co-enzyme Q10, EGCG, hydroxytyrosol and/or olive extract, shea butter, algae extract, cocoa butter, aloe extract, jojoba oil, echinacea extract, elastin, vitamin E and/or its derivatives, shea butter, algae extract, cocoa butter, aloe extract, panthenol and derivatives thereof, argan oil, collagen, saccharide isomerate, superoxide dismutase, calendula extract, edelweiss extract, glycine soja (soybean) protein, hydrolized rice protein, hypericum extract, linum extract, malva extract, marrubium extract, sambuccus extract, sericin, hydrolyzed sericin (Setakol), cephalins (Cephalipin), triticum vulgare (wheat) germ extract (Fitobroside), hyaluronic acid and salts thereof, glycoproteins, thyme extract, buddleja extract, imperatoria extract, plantago extract, saccharomyces cerevisiae extract, sambucus extract, ceratonia siliqua gum, ceramides, milk lipids, scutellaria extract, hyssopus extract, bisabolol, azulene.

The additional cosmetically active ingredient is typically included in an amount of at least 0.001 wt. % based on the total weight of the topical preparation. Generally, an amount of about 0.001 wt. % to about 30 wt. %, preferably from about 0.001 wt. % to about 10 wt. % of an additional cosmetically active agent is used.

Vitamin C (ascorbic acid) and/or its derivatives in particular ascorbyl phosphate such as Stay C (sodium ascorbyl monophosphate) is preferably used in the topical preparations according to the invention in an amount of 0.1-5 wt.-% in particular 0.1-2 wt.-%.

Shea butter is preferably used in the topical preparations according to the invention in an amount of 0.5-10 wt.-%, in particular 0.5-5 wt.-%.

Algae extract is preferably used in the topical preparations according to the invention in an amount of 0.1-10 wt.-%, in particular 0.5-1 wt.-%.

Aloe extract is preferably used in the topical preparations according to the invention in an amount of 0.1-10 wt.-%, in particular 0.5-1wt.-%.

Elastin is preferably used in the topical preparations according to the invention in an amount of 0.01-10 wt.-%, preferably 0.01-1 wt.-%

A vitamin E derivative for use in the topical preparations according to present invention is tocopheryl acetate. Tocopheryl acetate may be present in an amount from about 0.05 wt.-% to about 25 wt.-%, in particular 0.05 wt.-% to 5 wt.-% based on the total weight of the preparation. Another vitamin E derivative of interest is tocopheryl linoleate. Tocopheryl linoleate may be present in the topical preparations in an amount from about 0.05 wt.-% to about 25 wt.-% in particular 0.05 wt.-% to 5 wt.-%.

Vitamin A and/or its derivatives in particular retinoid derivatives such as retinyl palmitate or retinyl propionate is preferably used in the topical preparations according to the invention in an amount of 0.01-5 wt.-%, in particular 0.01-0.3 wt.-%

Cocoa butter is preferably used in the topical preparations according to the invention in an amount of 0.5-5 wt.-%.

Of course, one skilled in this art will take care to select the above mentioned optional additional compound or compounds and/or their amounts such that the advantageous properties intrinsically associated with the combination in accordance with the invention are not, or not substantially, detrimentally affected by the envisaged addition or additions.

Further preferred ingredients in the topical compositions according to the invention are pigments and colorants to diminish and to cover redness and blotches such as pigments and colorants conventionally used in make-up formulations.

The pigments according to invention can be inorganic or organic. Preferred ones in the sense of the present invention are pigment mixtures from white-pigments (e.g. Kaolin, titanium dioxide or zinc oxide) and inorganic color pigments (z. B. brown iron oxide pigments, chromium oxides), whereby the pigments may be coated or uncoated. As color pigments iron oxides are particularly preferred. Preferably, the white pigments do not show an absorption in the range of the visible light. Favourable according to the invention are white pigments such as e.g. titanium dioxides (refractive indexes: 2.55 for anatases and 2.75 for rutile) and zinc oxides (refractive index between 1.95 and 2.1). Particularly preferred is titanium dioxide.

Further pigments are gloss pigments which represent the most important group of the effect pigments such as e.g. Timiron of Merck, Iriodin of Merck (Perl and color gloss pigments for decorative technical applications), Xirallic of Merck (colorintense crystal effect pigments).

The topical preparations according to invention can also contain organic color pigments such as organic dyes, which are insoluble in the preparation such as e.g azo pigments and polycyclic pigments.

Furthermore it is favourable in the sense of the present invention, if the preparation according to invention contains one or several dyes whereas the dyes can be both of synthetic and natural origin.

Which amount of the topical preparation has to be applied, depends on the concentration of the active ingredient(s) in the product and the desired cosmetic effect(s). A typical “leave-on” composition like a skin care emulsion or a functional composition, for example, is usually applied in an amount of about 0.5 to about 2 mg per cm² skin. The applied amount is normally not critical, and the desired effect(s) may be achieved by using more of the composition, repeating the application of the composition and/or applying a composition which contains more of the active ingredient(s).

By “'leave-on' composition” as used herein a topical composition is meant which after having applied to the skin, is not removed intentionally. It is preferably left on the skin for a period of at least about 15 minutes, more preferably at least about 30 minutes, even more preferably at least about 1 hour, most preferably for at least several hours, e. g. up to about 12 hours.

In another embodiment, the invention also relates to a method of treatment or co-treatment of rosacea and its symptoms said method comprising the step of applying an effective amount of a topical preparation according to the invention with all the definition and preferences as given above to the skin of a subject in need of such a treatment. In particular, the invention relates to a method of treatment or co-treatment of skin redness, blushing, permanent erythema and telangiectasia, red small bumps and pimples as well as skin thickening said method comprising the step of applying an effective amount of a topical preparation with all the definition and preferences as given above to the skin of a subject in need of such a treatment.

Furthermore, the invention also relates to a method of treatment or co-treatment of blotchy skin, sensitive skin, dry skin, irritated skin, inflamed skin and atopic skin.

The term treatment or co-treatment as used in the present invention includes also a proactive use of the topical preparations in order to prevent any signs of rosacea and its symptoms.

An effective amount of a topical preparation in these methods refers to an amount necessary to obtain a physiological effect. The physiological effect may be achieved by one single dose or by repeated doses. The dosage administered may, of course, vary depending upon known factors, such as the physiological characteristics of the particular composition and its mode and route of administration; the age, health and weight of the recipient; the nature and extent of the symptoms; the kind of concurrent treatment; the frequency of treatment; and the effect desired and can be adjusted by a person skilled in the art. Preferably, the topical preparations are applied at least twice a day such as e.g. once in the morning and once in the evening.

The following examples are provided to further illustrate the compositions and effects of the present invention. These examples are illustrative only and are not intended to limit the scope of the invention in any way.

### EXAMPLE 1

A mixture of 41.4 wt.-% of Spent Grain Wax, 20 wt.-% conjugated linoleic acid (CLA, commercially available as Conjugated Linoleic Acid (CLA) 75% at Bioriginal, Netherlands), 5.64 wt.-% Behenic Acid, 20 wt.-% Syn-Coll® (comprising 0.02% Palmitoyl Tripeptide-5), 6 wt.-% Triolein, 3 wt.-% Linolic acid, 3 wt.-% Wheat germ oil, 0.75 wt.-% Palmitic acid, 0.13wt.-% Antioxidant and 0.08wt.-% Beta-Sitosterol was prepared which formed a stable W/O emulsion even upon storage.

### EXAMPLE 2

### Evaluation and Comparison of the Efficacy of Palmitoyl-Tripeptide 5 and CLA, Respectively Palmitoyl Tripeptide-5 and Spent Grain Wax Against the Symptoms of Rosacea

Three otherwise healthy Caucasians (female & male) aged 18+ displaying Rosacea of subtype 1 or 2 in the face treated twice a day with a composition as outlined in table 1. The affected skin areas on the face were cleansed before a thin layer (about 1-3 mg/cm2) of the composition (see Table 1) was gently massaged into the affected areas.

Visual assessment and biophysical measurement were collected prior to again after 7 and 14 days of use. Effect on telangiectasia and redness reduction assessed using Minolta CR-200 Chromameter interfaced with a DP-100 Color Computer System (Minolta CR-200).

TABLE 1
Formulation

| | | | 1 | 2 | 3 |
|---|---|---|---|---|---|
| | Trade Name | INCI | Wt.-% | | |
| A | Water | Water | 67.3 | 63.3 | 71.3 |
| | Hydrolite-5 | Pentylene Glycol | 3.0 | 3.0 | 3.0 |
| | Disodium | Disodium Phosphate | 0.2 | 0.2 | 0.2 |
| | Hydrogenphosphat | | | | |
| | anhydrous | | | | |
| | Sodium | Disodium Phosphate | 1.0 | 1.0 | 1.0 |
| | Hydrogenphosphat | | | | |
| | anhydrous | | | | |
| | SYN ®-COLL | Water, Glycerin, | 3.0 | 3.0 | — |
| | | Palmitoyl Tripeptide-5 | | | |
| B | Nat 8539 | Lecithin, Ethanol | 6.7 | 6.7 | 6.7 |
| C | Ethanol 96% | Ethanol | 10.0 | 10.0 | 10.0 |
| | Treberextrakt | Spent grain wax | — | 5.0 | 2.0 |
| | CLA-75% | Isomerized Safflower | 1.0 | — | — |
| | | Acid | | | |
| D | Glycerin | Glycerin | 2.0 | 2.0 | — |
| E | Phenonip | Phenoxyethanol | 0.3 | 0.3 | 0.3 |
| | | Methylparaben | | | |
| | | Ethylparaben | | | |
| | | Butylparaben | | | |
| | | Propylparaben | | | |
| | | Isobutylparaben | | | |
| F | HMW 2220 | Divinyldimethicone/ | 1.5 | 1.5 | 1.5 |
| | | Dimethicone Copolymer | | | |
| | | C12-13 Pareth-23 | | | |
| | | C12-13 Pareth-3 | | | |
| G | Novemer | Acrylates/Acrylamide | 4.0 | 4.0 | 4.0 |
| | EC-Polymer | Copolymer Mineral | | | |
| | | Oil Polysorbate 85 | | | |

TABLE 2
Results

| Tested | Redness Reduction % | Effect on Telangiectasia |
|---|---|---|
| substances | (Mean period of 2-4 weeks) | (Mean period of 2-4 weeks) |
| Formula 1 | 13.11 | 20.88 |
| Formula 2 | 10.92 | 41.63 |
| Formula 3 | −2.38 | −3.93 |
| (Control) | | |

As can be seen, the compositions according to the invention 1 and 2 shows a significant reduction in view of redness and telangiectasia compared to the control.

### EXAMPLE 3

### Reduction of Telangiectasia by Topical Application of a Composition Comprising a Composition According to Example 1 as Active Ingredient

40 otherwise healthy Caucasians (female & male) aged 18+ displaying Rosacea of subtype 1 or 2 in the face treated twice a day with a composition as outlined in table 3. The affected skin areas on the face were cleanses before a thin layer (about 1-3 mg/cm2) of the composition was gently massaged into the affected areas.

The surface of the telangiectasa was measured/assessed initially and after 42, respectively 85 days. The effect on telangiectasia was assessed using image analysis (digital photography NIKON 70S equipped with a NIKON 60 mm Macro objective) and the mean values over the 40 volunteers calculated. As can be seen in table 4, a significant reduction of telangiectasia has been observed.

TABLE 3
Formulation

| | | | 1 | 2 |
|---|---|---|---|---|
| | Trade Name | INCI | Wt.-% | |
| A | Water | Water | 66.3 | 66.3 |
| | Hydrolite-5 | Pentylene Glycol | 3.0 | 3.0 |
| | Disodium | Disodium Phosphate | 0.2 | 0.2 |
| | Hydrogenphosphat | | | |
| | anhydrous | | | |
| | Sodium | Sodium Phosphate | 1.0 | 1.0 |
| | Hydrogenphosphat | | | |
| | anhydrous | | | |
| B | Nat 8539 | Lecithin, Ethanol | 6.7 | 6.7 |
| C | Ethanol 96% | Ethanol | 10.0 | 10.0 |
| | Composition | | 2.0 | 5.0 |
| | according to | | | |
| | example 1 | | | |
| D | Glycerin | Glycerin | 2.0 | 4.0 |
| E | Phenonip | Phenoxyethanol Methylparaben | 0.3 | 0.3 |
| | | Ethylparaben Butylparaben | | |
| | | Propylparaben | | |
| | | Isobutylparaben | | |
| F | HMW 2220 | Divinyldimethicone/ | 1.5 | 1.5 |
| | | Dimethicone Copolymer | | |
| | | C12-13 Pareth-23 | | |
| | | C12-13 Pareth-3 | | |
| G | Novemer | Acrylates/Acrylamide | 4.0 | 4.0 |
| | EC-Polymer | Copolymer Mineral | | |
| | | Oil Polysorbate 85 | | |

TABLE 4
Results

| | Surface of | | | |
|---|---|---|---|---|
| | telangiectasia [mm²] | | | |
| | (mean values) | | | |
| | Day 0 | Day 42 | Day 84 | Overall reduction after 84 days |
| Formula 1 | 36.95 | 32.25 | 29.54 | −20% |
| Formula 2 | 40.46 | 31.44 | 30.82 | −28% |

As can be seen, the composition according to the invention significantly reduced the surface of telangiectasia.

### EXAMPLE 4

### Expression of IL-8, Respectively VEGF by Epidermal Human Skin Model After IL-1alpha Stimulation

The Expression of IL-8 by epidermal human skin model after IL-1alpha stimulation has been assessed using 3D-Reconstituted Epidermis Models (EST-1000) (Cellsystems GmbH, St. Katharinen, Germany). The EST-1000 samples were equilibrated over night at 37° C. in a 5% CO² atmosphere according to manufacturer's instructions. Then, the EST-1000 samples were stimulated by addition of 10 ng/ml IL-1alpha (Peprotech, UK) to the culture medium.

Afterwards, one sample was treated with the vehicle (i.e. squalane, e.g. commercially available under the tradename Fitoderm®), whereas the other sample was treated with a mixture consisting of the vehicle comprising 2 wt.-% of the mixture of example 1 by topical addition to the tissue samples. At distinct time points, 200 ul medium samples were taken and frozen for further analysis. As can be retrieved from the results presented in table, the expression of the pro-inflammatory cytokine IL-8 by the epidermal human skin model (EST-1000) is suppressed by the addition of the composition according to example 1 in a dose dependent manner. The expression of IL-8 is clearly induced by IL-1alpha after 4 hrs incubation. However the accumulation of IL-8 in the medium is significantly slower with 2% of the composition according to example 1 consisting essentially off spent grain wax, CLA and N2-(1-oxohexadecyl)-lysyl-valyl-lysine (Palmitoyl Tripeptide-5) compared to control showing a significant reduction in relation to skin irritation and skin inflammation.

TABLE 5

| | Expression of cytokine IL-8 | | | |
|---|---|---|---|---|
| | t = 0 h | t = 1 h | t = 4 h | t = 8 h |
| Control (Vehicle) | 47 | 45 | 887 | 1092 |
| 2 wt.-% of composition of | 16 | 46 | 373 | 749 |
| example 1 in vehicle | | | | |

The taken samples were also analyzed by EST-1000 in view of the expression of VEGF (Vascular Endothelial Growth Factor, an inducer of angiogenesis) which is used as a reference for reddening of the skin and telangiectasia (blood vessels). The expression of VEGF is suppressed by the addition of 2% of the composition according to example 1 consisting essentially off spent grain wax, CLA and N2-(1-oxohexadecyl)-lysyl-valyl-lysine (Palmitoyl Tripeptide-5) in a dose dependent manner translating into a significant reduction of reddening of the face and telangiectasia (Table 6).

TABLE 6

| | Expression of VEGF | | | |
|---|---|---|---|---|
| | t = 0 h | t = 1 h | t = 4 h | t = 8 h |
| Control (Vehicle) | 152 | 217 | 842 | 1698 |
| 2 wt.-% of composition of | 60 | 289 | 389 | 658 |
| example 1 in vehicle | | | | |

Furthermore, the expression of IL-8 by epidermal human skin model after IL-1alpha stimulation was assessed as outlined above using the single compounds CLA, Syn Coll®, spent grain wax as well as mixtures thereof. As can be retrieved from table 7, the accumulation of IL-8 in the medium comprising 2 wt.-% of a 1:1 mixture of CLA and Syn Coll® is significantly slower compared to the single compounds thus showing a synergistic effect in relation to skin irritation and skin inflammation.

TABLE 7

| | Expression of cytokine IL-8 | | | | |
|---|---|---|---|---|---|
| | t = 0 h | t = 2 h | t = 4 h | t = 8 h | T = 24 h |
| Control (Vehicle) | 58 | 2219 | 5021 | 6147 | 6806.5 |
| 2 wt.-% CLA in | 61 | 2536 | 5056 | 5805 | 8161 |
| vehicle | | | | | |
| 2 wt.-% Syn Coll ® | 65 | 303 | 1070 | 2747 | 5791.5 |
| in vehicle | | | | | |
| 2 wt.-% Spent grain | 59 | 3007 | 5849 | 6448 | 6891 |
| wax in vehicle | | | | | |
| 2 wt.-% Syn Coll ®/ | 62 | 269 | 1079 | 2190 | 3282 |
| CLA (1:1) in vehicle | | | | | |

Furthermore, the expression of VEGF by epidermal human skin model after IL-1alpha stimulation was assessed as outlined above using mixtures of spent grain wax and CLA, spent grain wax and Syn Coll® and spent grain wax, CLA and Syn Coll®. As can be retrieved from table 8, the expression of VEGF is suppressed by the addition of 2 wt.-% of the respective composition translating into a significant reduction of reddening of the face and telangiectasia.

TABLE 8

| | Expression of VEGF | | | | |
|---|---|---|---|---|---|
| | t = 0 h | t = 2 h | t = 4 h | t = 8 h | t = 24 h |
| Control (Vehicle) | 20 | 126.5 | 201 | 304 | 1058 |
| 2 wt.-% of spent grain | 0 | 114 | 208 | 375 | 752.5 |
| wax & CLA (2.75:1) in | | | | | |
| vehicle | | | | | |
| 2% of spent grain wax | 25 | 95 | 211 | 408 | 851 |
| and Syn Coll (2.75:1) | | | | | |
| in vehicle | | | | | |
| 2 wt.-% of spent grain | 30.5 | 120 | 224 | 360 | 621 |
| wax, CLA & Syn Coll | | | | | |
| (2.75:1:1) in vehicle | | | | | |

### EXAMPLE 5

### Stabilization of W/O Emulsion Pre-Mixes Comprising N2-(1-oxohexadecyl)-lysyl-valyl-lysine, Spent Grain Wax and Conjugated Linoleic Acid

| | | | |
|---|---|---|---|
| Spent grain wax | 35.0% | 35.0% | 35.0% |
| CLA | 20.0% | 20.0% | 20.0% |
| Water | Ad 100% | Ad 100% | Ad 100% |
| SYN ®-COLL | 17.5% | 17.5% | 17.5% |
| (comprising 0.02% Palmitoyl | | | |
| Tripeptide-5) | | | |
| STIMU-TEX ® | 15.0% | 25.0% | 25.0% |
| Palmitic acid | 5.0% | 0.0% | 0.0% |
| Behenic acid | 0.0% | 5.0% | 0.0% |
| Emulsifier* | 0.0% | 0.0% | 5.0% |
| Stability | Not stable | Stable | Not stable |

*Tested were emulsifiers like Olivem 1000 (O/W), Olivem 900 (W/O) (B&T Srl., Italy) Phospholipon 80 H and Phospholipon 85 G (Phospholipid GmbH, Germany) and Oliwax (B&T Srl., Italy) as a stabilizer

Different W/O formulations were prepared and stored. The stability of the emulsions was assessed for at least 3 months at 4°, 20° and 40°. Surprisingly, neither the use of palmitic acid nor an emulsifier yielded in a stable emulsion, whereas the addition of behenic acid resulted in a stable pre-mix composition.

### EXAMPLE 6

### Rosacea Day Cream

| Phase | Ingredients | INCI Name | % Wt. |
|---|---|---|---|
| A | Butylene Glycol | Butylene Glycol | 2.50 |
| | Timiron Silk Green | C.I. 77891 | 3.00 |
| | J-68-BC | Talc | 3.00 |
| | Sun CHROMA Hydrated | C.I. 77289 | 0.50 |
| | Chrome Oxide Green C | | |
| | 61-1245 | | |
| B | Dervacid 3155 Flake | Stearic Acid | 2.50 |
| | Arlacel 165 | Glyceryl Stearate, PEG-100 | 2.00 |
| | | Stearate | |
| | Myritol 318 | Caprylic/Capric | 3.00 |
| | | Triglyceride | |
| | Eutanol G | Octyldodecanol | 3.40 |
| | Parsol SLX | Polysilicone-15 | 1.00 |
| | Parsol 1789 | Butyl | 1.00 |
| | | Methoxydibenzoylmethane | |
| | Parsol 340 | Octocrylene | 0.80 |
| | DC 345 | Cyclopentasiloxane | 5.00 |
| | | Cyclohexasiloxane | |
| | DC 556 | Phenyl Trimethicone | 2.00 |
| C | Water deionized | Aqua | Add 100 |
| | Butylene Glycol | Butylene Glycol | 2.50 |
| | Euxyl PE 9010 | Phenoxyethanol, | 1.00 |
| | | Ethylhexylglycerin | |
| D | SYN ®-TACKS | Glycerin (and) Palmitoyl | 3.00 |
| | | Dipeptide-5 | |
| | | Diaminobutyroyl | |
| | | Hydroxythreonine (and) | |
| | | Palmitoyl Dipeptide-5 | |
| | | Diaminohydroxybutyrate | |
| | Composition according | Spent Grain Wax, | 3.00 |
| | to example 1 | Linoleic Acid, Behenic | |
| | | Acid, Palmitoyl | |
| | | Tripeptide-5 | |
| E | NaOH | Sodium hydroxide | q.s. |

Pre-blend phase A. Heat phase B to 80° C. Heat phase C to 80° C. Add phase B to phase C using high shear mixing. Add phase A to the batch. Add phase D to the batch. Adjust pH to pH 5.0-5.5 using phase E as necessary.

### EXAMPLE 7

### Night Cream

| Phase | Ingredients | INCI Name | % Wt. |
|---|---|---|---|
| A | Imwitor 372P | Glyceryl Stearate Citrate | 2.00 |
| | Cutina GMS | Glyceryl Stearate | 3.00 |
| | Sympatens-O/4200 | Sorbitan Laurate & | 1.00 |
| | | Polyglyceryl-10 | |
| | Sweet Almond Oil | Prunus Amygdalus Dulcis | 2.50 |
| | | (Sweet Almond) Oil | |
| | Tegosoft TN | C12-15 Alkyl Benzoate | 7.00 |
| | Cetiol OE | Dicaprylyl Ether | 5.00 |
| | Tegosoft DC | Decyl Cocoate | 3.00 |
| | Mixed Tocopherols 95 | Tocopherol | 0.50 |
| | Euxyl PE 9010 | Phenoxyethanol, | 0.80 |
| | | Ethylhexylglycerin | |
| | Dow Corning 345 | Cyclopentasiloxane, | 2.00 |
| | | Cyclohexasiloxane | |
| B | Keltrol RD | Xanthan Gum | 0.30 |
| | Glycerin | Glycerin | 2.00 |
| | Wasser ad 100 | Aqua | 57.90 |
| | ROPUFA ‘10’ N-6 Oil | Oenothera Biennis (Evening | 3.00 |
| | | Primrose) Oil (and) | |
| | | Tocopherol (and) | |
| | | Ascorbyl Palmitate | |
| | Composition according | Spent Grain Wax, Linoleic | 4.00 |
| | to example 1 | Acid, Behenic Acid, Palmitoyl | |
| | | Tripeptide-5 | |
| | SYN ®-TACKS | Glycerin (and) Palmitoyl | 3.00 |
| | | Dipeptide-5 Diaminobutyroyl | |
| | | Hydroxythreonine (and) | |
| | | Palmitoyl Dipeptide-5 | |
| | | Diaminohydroxybutyrate | |
| | ALPAFLOR ® | Glycerin (and) Water (and) | 3.00 |
| | EDELWEISS GC | Leontopodium Alpinum Extract | |
| C | NaOH | Sodium hydroxide | q.s. |

Mix phase A until homogenous. Add phase B to phase A mixing thoroughly. Adjust pH to pH 5.0-5.5 using phase C as necessary. Add phase D mixing thoroughly between each addition.

### EXAMPLE 8

### Concealer

| Phase | Ingredients | INCI Name | % Wt. |
|---|---|---|---|
| A | J-68-BC | Talc | 3.00 |
| | DC 9701 Cosmetic | Dimethicone/Vinyl | 2.00 |
| | Powder | Dimethicone Crosspolymer | |
| | | Silica | |
| | Timiron Silk Green | Titanium Dioxide Mica | 3.00 |
| | | C.I. 77891 | |
| | Suncroma Chromoxide | C.I. 77288 | 0.50 |
| | green | | |
| | Suncroma ultramarine | C.I 77007 | 0.05 |
| | blue C43-1810 | | |
| B | Cetiol CC | Dicaprylyl Carbonate | 2.00 |
| | DC 556 Fluid | Phenyl Trimethicone | 2.40 |
| | LexFeel 7 | Neopentyl Glycol | 3.40 |
| | | Diheptanoate | |
| | Parsol SLX | Polysilicone-15 | 1.00 |
| | Parsol 1789 | Butyl | 1.00 |
| | | Methoxydibenzoylmethane | |
| | Parsol 340 | Octocrylene | 0.80 |
| C | DC 9040 | Cyclopentasiloxane, | Add 100 |
| | | Dimethicone Crosspolymer | |
| | DC BY 11-030 | Cyclopentasiloxane, | 5.00 |
| | Emulsifier G | PEG/PPG-19/19 Dimethicone | |
| | DC 345 | Cyclopentasiloxane | 3.00 |
| | | Cyclohexasiloxane | |
| | Euxyl 9010 | Phenoxyethanol | 0.80 |
| | | Ethylhexylglycerin | |
| D | PREREGEN ® | Glycine Soja (Soybean) | 2.00 |
| | | Protein (and) Oxido | |
| | | Reductases | |
| | Composition according | Spent Grain Wax, Linoleic | 3.00 |
| | to example 1 | Acid, Behenic Acid, | |
| | | Palmitoyl Tripeptide-5 | |

Pre blend phase A. Heat phase B to 75°. Heat phase C to 50°. Add phase B to phase C Add phase A to the batch. Add phase D to the batch.

### EXAMPLE 9

### Intensive Serum

| Phase | Ingredients | INCI Name | % Wt. |
|---|---|---|---|
| A | RapiThix A60 | Sodium Polyacrylate (and) | 2.00 |
| | | Hydrogenated Polydecene | |
| | | (and) Trideceth-6 | |
| B | Water deionized | Aqua | Ad 100 |
| | Lara Care A200 | Arabinogalactan | 0.20 |
| | Eumulgin L | PPG-1-PEG-9 Lauryl | 1.50 |
| | | Glycol Ether | |
| | Glycerin | Glycerin | 5.00 |
| | Euxyl PE 9010 | Phenoxyethanol, | 0.80 |
| | | Ethylhexylglycerin | |
| | Composition according | Spent Grain Wax, Linoleic | 5.00 |
| | to example 1 | Acid, Behenic Acid, | |
| | | Palmitoyl Tripeptide-5 | |
| C | ELHIBIN ® | Glycine Soja (Soybean) | 2.00 |
| | | Protein | |
| | PENTAVITIN ® | Saccharide Isomerate | 2.00 |
| | NaOH 10% | Sodium Hydroxide | q.s. |

Mix Hispagel and water together until homogenous. Incorporate the other components one at a time. Mix until homogenous. Adjust pH with phase C.

### EXAMPLE 10

### Cream Gel

| Phase | Ingredients | INCI Name | % Wt. |
|---|---|---|---|
| A | Water deion. | Aqua | 70.80 |
| | DC 200, 0.65cs | Trisiloxane | 0.05 |
| | Carbopol Ultrez 21 | Acrylates/C10-30 Alkyl Acrylate | 0.25 |
| | | Crosspolymer | |
| B | Glycerin | Glycerin | 3.00 |
| | Euxyl PE 9010 | Phenoxyethanol, | 0.80 |
| | | Ethylhexylglycerin | |
| C | Novemer EC-1 | Acrylates/Acrylamide | 2.00 |
| | Polymer | Copolymer (and) Mineral Oil | |
| | | (and) Polysorbate 85 | |
| | Lexfeel 7 | Neopentyl Glycol Diheptanoate | 5.00 |
| | Tegosoft CI | Cetearyl Isononanoate | 3.00 |
| | Merquat Plus 3330 | Polyquaternium 39 | 2.00 |
| | DC 345 | Cyclopentasiloxane, | 2.00 |
| | | Cyclohexasiloxane | |
| D | NaOH 10% | Sodium Hydroxide | q.s. |
| E | Composition accordin | Spent Grain Wax, Linoleic | 5.00 |
| | example 1 | Acid, Behenic Acid, | |
| | | Palmitoyl Tripeptide-5 | |
| | PREREGEN ® | Glycine Soja (Soybean) Protein | 3.00 |
| | | (and) Oxido Reductases | |
| | PEPHA ®-PROTECT | Water (and) Glycerin (and) | 3.00 |
| | | Citrullus Lanatus (Watermelon) | |
| | | Fruit Extract | |
| | Parfum | Parfum | 0.10 |

Mix phase A until homogenous. Add phase B to phase A mixing thoroughly. Add phase C to the batch mixing thoroughly. Adjust pH using phase D as necessary. Add phase E mixing thoroughly between each addition

### EXAMPLE 11

### Oil Bath Water Free

| Phase | Ingredients | INCI Name | % Wt. |
|---|---|---|---|
| A | Zetesol TP 200 | TIPA-Laureth Sulfate (and) | 34.00 |
| | | Propylene Glycol | |
| | Oxetal VD 20 | Laureth-20 | 23.00 |
| | Pluronic PE 3100 | Poloxamer 101 | 1.00 |
| | Sunflower oil | Helianthus Annuus (Sunflower) | 26.50 |
| | | Seed Oil | |
| | ROPUFA ® | Borago Officinalis Seed Oil | 10.00 |
| | ‘25’ N-6 Oil | (and) Tocopherol (and) | |
| | | Ascorbyl Palmitate | |
| | Mixed Tocopherols | Tocopherol | 0.50 |
| | Composition | Spent Grain Wax, Linoleic Acid, | 5.00 |
| | according to | Behenic Acid, Palmitoyl | |
| | example 1 | Tripeptide-5 | |

Mix phase A until homogenous

### EXAMPLE 12

### Baby Wash and Make-Up Remover

| Phase | Ingredients | INCI Name | % Wt. |
|---|---|---|---|
| A | Plantapon SF | Sodium Cocoamphoacetate | 20.00 |
| | | Glycerin Lauryl Glucoside | |
| | | Sodium Cocoyl Glutamate | |
| | | Sodium Lauryl Glucose | |
| | | Carboxylate | |
| | Gluadin WQTM | Hydroxypropyltrimonium | 1.00 |
| | | Hydrolyzed Wheat Protein | |
| B | Water deionized | Aqua | Ad 100 |
| | Euxyl PE 9010 | Phenoxyethanol, | 0.80 |
| | | Ethylhexylglycerin | |
| | Mixed Tocopherols | Tocopherol | 0.80 |
| | Panthenol 75L | Panthenol | 2.00 |
| | Pentavitin ® | Saccharide Isomerate | 3.00 |
| | Composition according | Spent Grain Wax, Linoleic Acid, | 2.00 |
| | to example 1 | Behenic Acid, Palmitoyl | |
| | | Tripeptide-5 | |
| C | NaOH | Sodium hydroxide | q.s. |

Mix phase A until homogenous. Add phase B to phase A mixing thoroughly. Adjust pH to pH 5.0-5.5

### EXAMPLE 13

### Baby Cream

| Phase | Ingredients | INCI Name | % Wt. |
|---|---|---|---|
| A | Zinc Oxide | Zinc Oxide | 15.00 |
| | Lanette O | Cetearyl Alcohol | 3.50 |
| | Dehymuls PGPH | Polyglyceryl-2 | 2.00 |
| | | Dipolyhydroxystearate | |
| | Eumulgin B 2 | Ceteareth-20 | 2.50 |
| | Isohexadecan | Isohexadecane | 8.00 |
| | Tegosoft M | Isopropyl Myristate | 5.00 |
| | Mixed Tocopherols 95 | Tocopherol | 0.50 |
| | Euxyl PE 9010 | Phenoxyethanol, | 0.80 |
| | | Ethylhexylglycerin | |
| | Dow Corning 345 | Cyclopentasiloxane, | 2.00 |
| | | Cyclohexasiloxane | |
| B | Keltrol RD | Xanthan Gum | 0.30 |
| | Glycerin | Glycerin | 2.00 |
| | Water deionized | Aqua | Ad 100 |
| | Panthenol 75L | Panthenol | 2.00 |
| | Magnesium Sulfate | Magnesium Sulfate | 1.00 |
| | Heptahydrate | | |
| | Natriummetabisulfit | Sodium Metabisulfite | 0.03 |
| | Composition according | Spent Grain Wax, Linoleic Acid, | 2.00 |
| | to example 1 | Behenic Acid, Palmitoyl | |
| | | Tripeptide-5 | |
| | Allantoin | Allantoin | 0.50 |
| | Alpalor ® Malva AO | Malva sylvestris (Mallow) | 3.00 |
| | | flower extract, Glycerin, | |
| | | Water | |
| | Pentavitin ® | Saccharide Isomerate | 3.00 |
| C | NaOH | Sodium hydroxide | q.s. |

Mix phase A until homogenous. Add phase B to phase A mixing thoroughly. Adjust pH to pH 5.0-5.5using phase C as necessary. Add phase D mixing thoroughly between each addition.

### EXAMPLE 14

### Liquid Foundation

| Phase | Ingredients | INCI Name | % Wt. |
|---|---|---|---|
| A | Wacker Belsil MM 8030 | C30-45 Alkyl Methicone | 2.70 |
| | VP | | |
| | Wacker Belsil SPG | Cyclopentasiloxane, | 10.00 |
| | 128 VP | Caprylyl Dimethicone | |
| | | Ethoxy Glucoside | |
| | Ceraphyl 494 | Isocetyl Stearate | 0.30 |
| | Isohexadecan | Isohexadecane | 9.00 |
| | Hostacerin DGI | Polyglyceryl-2- | 2.50 |
| | | Sesquiisostearate | |
| | Wacker Belsil TMS 803 | Trimethylsiloxysilicate | 1.50 |
| | Parsol SLX | Polysilicone-15 | 1.20 |
| | Parsol 1789 | Butyl | 1.30 |
| | | Methoxydibenzoylmethane | |
| | Parsol 340 | Octocrylene | 0.90 |
| B | Unipure Red LC 304 | CI 15850 | 0.30 |
| | Unipure Brown LC 881 | CI 77491, CI 77492, | 0.30 |
| | | CI 77499 | |
| | Unipure Black LC 989 | CI 77499 | 0.05 |
| | Pigment Jaune W 1801 | Iron Oxides | 1.00 |
| | J-68-BC | Talc | 5.00 |
| C | Water deionized | Aqua | Add 100 |
| | Magnesium Sulfate | Magnesium Sulfate | 1.00 |
| | Heptahydrate | | |
| | Composition according | Spent Grain Wax, Linoleic | 3.00 |
| | to example 1 | Acid, Behenic Acid, | |
| | | Palmitoyl Tripeptide-5 | |
| | Allantoin | Allantoin | 0.50 |
| | Pentavitin ® | Saccharide Isomerate | 2.00 |
| | Euxyl PE 9010 | Phenoxyethanol, | 0.80 |
| | | Ethylhexylglycerin | |
| | Mixed Tocopherols | Tocopherol | 0.80 |

Heat phase A and C to 85° C. Add phase B to phase A while homogenizing. Add C while homogenizing. Cool down under stirring to room temperature.

### EXAMPLE 15

### Eye Gel

| Phase | Ingredients | INCI Name | % Wt. |
|---|---|---|---|
| A | Water deionized | Aqua | Ad 100 |
| | Glycerin | Glycerin | 3.00 |
| | Preservative | Preservative | q.s. |
| | Carbopol Ultrez 21 | Acrylates/C10-30 Alkyl Acrylate | 0.45 |
| | | Crosspolymer | |
| B | NaOH | Sodium hydroxide | q.s. |
| C | REGU ®-AGE | Hydrolyzed Rice Bran Protein, | 3.00 |
| | | Glycine Soja (Soybean) | |
| | | Protein, Oxido Reductases | |
| | Composition according | Spent Grain Wax, Linoleic Acid, | 3.00 |
| | to example 1 | Behenic Acid, | |
| | | Palmitoyl Tripeptide-5 | |

Mix phase A until homogenous. Adjust pH to pH 5.0-5.5 using phase B. Add phase C under stirring.

1. Composition comprising at least two compounds selected from N2-(1-oxohexadecyl)-lysyl-valyl-lysine, spent grain wax and/or conjugated linoleic acid.

2. A composition according to claim 1 comprising N2-(1-oxohexadecyl)-lysyl-valyl-lysine, spent grain wax and conjugated linoleic acid.

3. A composition according to claim 1, wherein the N2-(1-oxohexadecyl)-lysyl-valyl-lysine is in the form of the bistrifluoroacetate salt of N2-(1-oxohexadecyl)-L-lysyl-L-valyl-L-lysine.

4. A composition according to claim 1, wherein the conjugated linoleic acid is an isomeric mixture of cis-9-trans-11 and trans-10-cis-12 isomers.

5. A composition according to claim 1, characterized in that the composition is a pre-mix comprising from 0.01 to 0.05 wt.-% of N2-(1-oxohexadecyl)-lysyl-valyl-lysine, from 30-50 wt. -% of spent grain wax and from 15-25 wt.-% of conjugated linoleic acid, 1-10 wt.-% water and 5-35 wt.-% of glycerin.

6. A pre-mix according to claim 5, characterized in that as further ingredient behenic acid is present in an amount of 3-7 wt.-%, preferably 5 to 6 wt.-%.

7. A composition according to claim 1, characterized in that the composition is a topical preparation further comprising a cosmetically acceptable carrier.

8. A topical preparation according to claim 7 comprising from about 0.00002 to 0.002 wt.-% of N2-(1-oxohexadecyl)-lysyl-valyl-lysine, from about 0.04 to 4 wt.-% of conjugated linoleic acid and from about 0.02 to 2 wt.-% of spent grain wax based on the total weight of the topical preparation.

9. A topical preparation according to claim 8 obtainable by admixing a pre-mix into a cosmetically acceptable carrier.

10. A topical composition according to claim 7 which is in the form of an O/W emulsion, W/O emulsion, gel, surfactant mixture, Si/Si emulsion, Si/W emulsion, W/Si emulsion or a solution.

11. A topical composition according to claim 7, further comprising soothing ingredients selected from panthenol, bisabolol and/or azulene.

12. A topical composition according to claim 7, further comprising a pigment and/or colorant.

13. A composition according to claim 1 for the treatment or co-treatment of rosacea and its symptoms.

14. A composition according to claim 1 for the treatment or co-treatment of skin redness, blushing and telangiectasia.

15. A composition according to claim 1 for treatment or co-treatment of blotchy skin, sensitive skin, dry skin, irritated skin, inflamed skin, atopic skin.

16. A method of treatment or co-treatment of rosacea and its symptoms said method comprising the step of applying an effective amount of a topical preparation according to claim 6 to the skin of a subject in need of such a treatment.

17. A method according to claim 16 for the treatment or co-treatment of skin redness, blushing, telangiectasia, blotchy skin, sensitive skin, dry skin, irritated skin, inflamed skin and atopic skin.
